(19) United States
(12) Patent Application Publication
(10) Pub. No.: US 2017/0042633 A1
(43) Pub. Date: Feb. 16, 2017

(54) Tissue Marking For Lesion Removal

(71) Applicant: Malignext Targeting Technologies, Inc., Vancouver, WA (US)

(72) Inventors: Jarrett LeClaire, Vancouver, WA (US); Jamie Khaw, Portland, OR (US)

(73) Assignee: Malignext Targeting Technologies, Inc., Vancouver, WA (US)

(21) Appl. No.: 15/231,781

(22) Filed: Aug. 9, 2016

Related U.S. Application Data
(63) Continuation of application No. 12/795,081, filed on Jun. 7, 2010.

(51) Int. Cl. A61B 90/00

(52) U.S. Cl. CPC A61B 90/39; A61B 2090/395; A61B 2090/3933; A61B 2090/3966; A61B 2090/3925; A61B 2090/3908; A61B 2090/3987

(57) ABSTRACT

A preoperative tumor marking may be used to assist physicians in the removal of tumor tissue by clearly defining the margins of the tumor. An imaging technique, such as mammography, may be employed to visualize the tumor. The visualized tumor may be marked with a polymer, either directly or indirectly. The polymer may be liquid at room temperature and harden at body temperature, making the tumor palpable, but not changing the tumor's cellular and physical state. The polymer may also include a coloring agent that may aid in visualizing the lesion. This will assist a surgeon during excision by allowing him/her to both feel and see the tumor and remove enough of the tissue surrounding the tumor to greatly reduce the need for a second surgical procedure due to a failure to remove all tumor tissue during an initial surgery.

### BACKGROUND

Breast cancer will be diagnosed in approximately one in eight women in their lifetime and one in 30 will die of the disease. It is the leading cause of cancer deaths in women 40-55 years of age and the second leading cause of cancer deaths in women overall.

In order to treat and prevent breast cancer, it is often desirable and necessary to detect and test tissue abnormalities for pre-malignant conditions. This is particularly important in the diagnosis and treatment of patients with cancerous tumors, or lesions. Typically, a medical professional will diagnose the existence of suspicious circumstances by palpation, mammography, x-ray, MRI, or ultrasound imaging. The medical professional will then collect a biopsy to determine whether the tissue is cancerous.

If removal is recommended as a viable treatment, then the tumor or lesion will be marked to facilitate its removal during surgery. Marking prior to surgery is required because some tumors, such as Ductal Carcinoma in Situ (DCIS), are not palpable which makes locating the lesion during surgery problematic because the physician cannot feel the tumor. A number of procedures and devices for marking and locating particular tissue locations are known. For example, wire guides may be used for locating lesions. The wire guides have a tubular introducer needle and an attached wire guide with a helical coil configuration for locking into position once inside or near the targeted lesion.

The medical professional locates the lesion site using an imaging system, for example, x-ray, ultrasound or magnetic resonance imaging (MRI), in order to deploy the wire guides in or around the lesion. The needle may then be removed from the wire guide which remains locked in the lesion for guiding a surgeon down the wire to the lesion site during subsequent surgery. Wire guides have some disadvantages including inability to mark the entire lesion, and inability to mark the lesion in three dimensions.

Using the existing techniques for locating the lesions to be removed physicians can locate and remove cancerous tissue. However, for some types of cancer such as DCIS the national reexision rate, the cases in which the surgeon must do another surgery to completely remove the lesion, is around 40-50%. This is because the surgeon may fail to remove enough tissue around the lesion, the “margin,” and as a result leave cancerous cells within the patient. Inadequate excision of cancer-free margin of tissue is known to increase the risk of local recurrence of breast cancer. Alternatively, the physician may remove more tissue than is necessary. There is a need for improved techniques to ensure that the entire lesion can be marked, ensure that sufficient margin is removed, and thus decrease the need for reexcision.

### SUMMARY

A poloxamer, or hydrogel such as poly(N-isopropylacrylamide) can be used to effectively mark a lesion in preparation for surgery. When Poly(N-isopropylacrylamide) increases in temperature above a transition temperature it undergoes a reversible phase change from liquid phase to solid phase.

During visualization of the lesion using a technique such as mammography, the polymer may be injected directly into the lesion. Alternatively, the polymer may be injected around the tumor. The polymer permeates the tissue, leaving it unchanged except for the presence of the polymer and hardens as it approaches a transition temperature of 37 degrees Celsius. Following polymerization the lesion will be palpable and will increase the chances of a complete excision.

The necessary items for performing the methods of the invention can be included in a kit. The kit may include the temperature responsive hydrogel, a syringe that is at least partially insulated and an insulated needle. The hydrogel comprises a poloaxomer such as poly(N-isopropylacrylamide) and will undergo a reversible phase change at a transition temperature. The kit can also include a temperature controlling device to keep the polymer below the transition temperature and in a liquid state. The polymer may be contained in a carpule to be loaded into the syringe, or may be stored in the syringe.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items.

FIG. 1 is a view of an injection of a temperature responsive polymer into a lesion within a tissue.

FIG. 2 is a view of an injection of a temperature responsive polymer into tissue surrounding a lesion.

FIG. 3 is a view of an excision of a marked lesion.

FIG. 4 is a view of a temperature responsive polymer draining from an excised lesion.

FIG. 5 is a view of a lesion marked with a temperature responsive polymer containing beads.

FIG. 6 is a view of a medical professional injecting a temperature responsive polymer into a patient.

FIG. 7 is a view of a medical professional removing a lesion marked with a temperature responsive polymer from a patient.

FIG. 8 is a photograph of a chicken breast being injected with a temperature responsive polymer.

FIG. 9 is a photograph of a dissected chicken breast following injection with a temperature responsive polymer.

### DETAILED DESCRIPTION

This application describes tissue marking for lesion removal. The lesion removal includes identifying a region containing an area of interest and then marking the area with a temperature responsive hydrogel/polymer. The polymer undergoes a reversible phase change from liquid to solid when a transition temperature is reached. The polymer may make the tissue easier to detect by changing density of the tissue, marking the tissue with a color, making the tissue radio-opaque, and the like. However, the polymer does not change any aspect of the tissue related to diagnosis or analysis of the tissue.

By making the area of interest palpable to a medical professional the area can then be removed for further study and to prevent spread of potentially cancerous cells. This will benefit individuals that require a surgery to remove tissue as part of the treatment by helping a medical professional remove all affected tissue thus decreasing the chance that another surgery will be necessary.

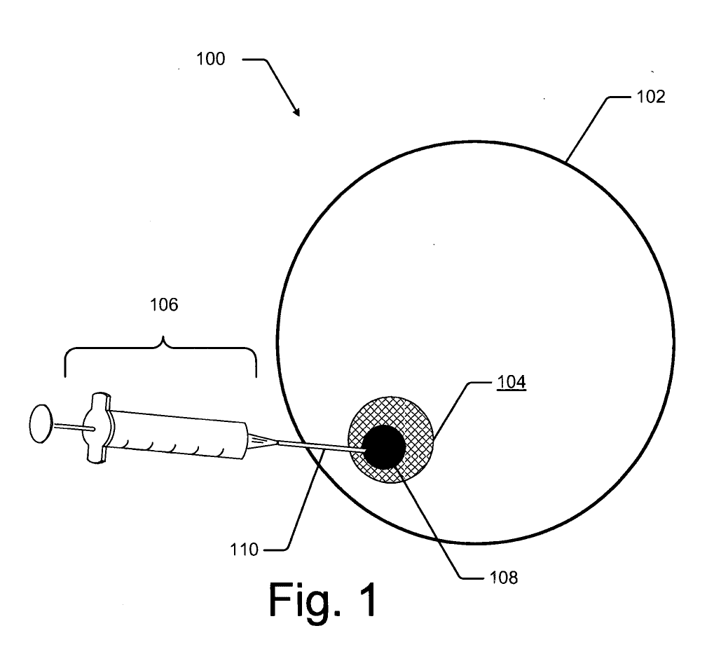

FIG. 1 is an illustration 100 of marking tissue 102 containing a lesion 104 in situ. The tissue 102 may include any form of tissue found in the body such as kidney, lung, stomach, liver, but, in one implementation the tissue 102 is breast tissue. The lesion 104 could include any type of atypical or cancerous lesion which may be palpable or unpalpable. In one implementation the lesion 104 is ductal carcinoma in situ (DCIS).

The lesion 104 is identified and visualized using a technique such as mammography, sonography, magnetic resonance imaging, breast specific gamma imaging, thermography, or the like. The polymer 108 is then delivered to the site of the lesion 104. A syringe 106 may be used to deliver the polymer 108 through the epidermis of a patient into the lesion 104 by placing a tip of a needle 110 at the location of the lesion 104.

The polymer 108 may be selected from the family of hydrogels which include poloaxomers and poly(N-isopropylacrylamide), and undergoes a reversible phase change, from liquid to solid, when the polymer 108 is heated up to the in situ temperature of the breast tissue 102 containing the lesion 104.

The properties of the breast tissue 102 and the lesion 104 remain unchanged after injection of the polymer 108 and the polymer 108 will permeate the lesion 104 making the lesion palpable once the polymer 108 reaches a transition temperature. Making the lesion 104 palpable will facilitate its excision by a medical professional. To protect the polymer 108 from reaching the transition temperature prematurely the syringe 106 may be at least partially insulated and the needle 110 may also be insulated.

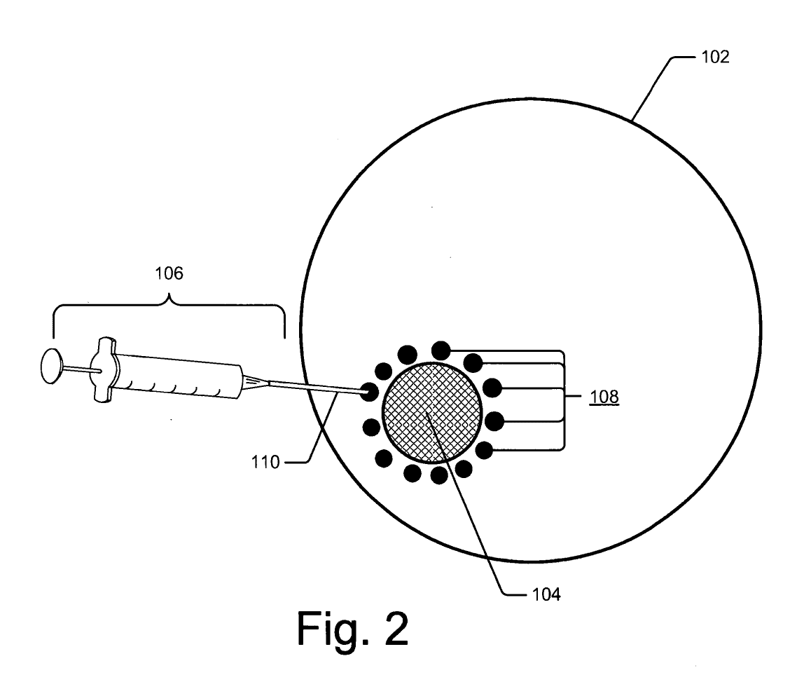

FIG. 2 is an illustration 200 of a different technique to mark tissue 102 containing an unpalpable lesion 104 in situ. In one implementation, the tissue 102 may be breast tissue but is not limited to one particular type of tissue. The lesion 104 in one implementation may be a cancerous tumor, such as DCIS, which is not palpable but may be identified and visualized using an imaging technique such as mammography, sonography, magnetic resonance imaging, or thermography.

The polymer 108 is then delivered to the area of interest. A syringe 106 may be used to deliver the temperature responsive polymer 108 into the tissue 102 surrounding the lesion 104 forming a coating around the lesion 104 that may encompass sufficient margins to aid in the removal of all potentially atypical and/or cancerous cells.

The polymer 108 may be a one or more hydrogels including poloaxomers and poly(N-isopropylacrylamide) the undergoe a reversible phase change, from liquid to solid, when the polymer 108 is heated up above the transition temperature to the in situ temperature of the breast tissue 102.

The properties of the breast tissue 102 and lesion 104 remain unchanged after injection of the polymer 108 and the polymer 108 will permeate the tissue 102 surrounding the lesion 104 making the lesion 104 palpable and facilitating its excision by a medical professional.

Similar to the technique illustrated in FIG. 1, it may be desirable to prevent the polymer 108 from reaching the transition temperature before being injected. To protect the polymer 108 from reaching the transition temperature prematurely, the syringe 106 may be at least partially insulated and the needle 110 may also be insulated.

The polymer 108 may also be precooled so that during transportation or distribution the polymer 108 will remain in its liquid state. This can be accomplished by the use of traditional coolants, which may include wet ice, dry ice, insulation, or the like.

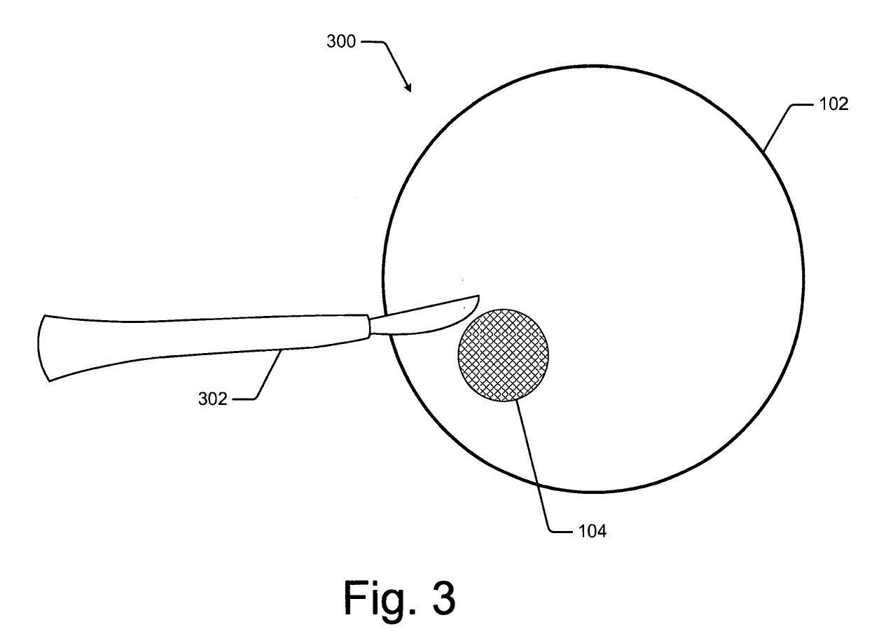

FIG. 3 is an illustration 300 of excising the lesion 104 from the tissue 102. The location of the lesion 104 within the breast tissue 102 may be marked by either of the techniques illustrated in FIG. 1 or 2 and it then excised by a medical professional. In one non-limiting implementation the medical professional uses a scalpel 302 to free the lesion 104 from the surrounding tissue; however, other techniques could be conceivably used to excise the lesion 104 from the tissue 102. For example, other sharp or cautery dissections techniques including knives, scissors, and cautery/heat.

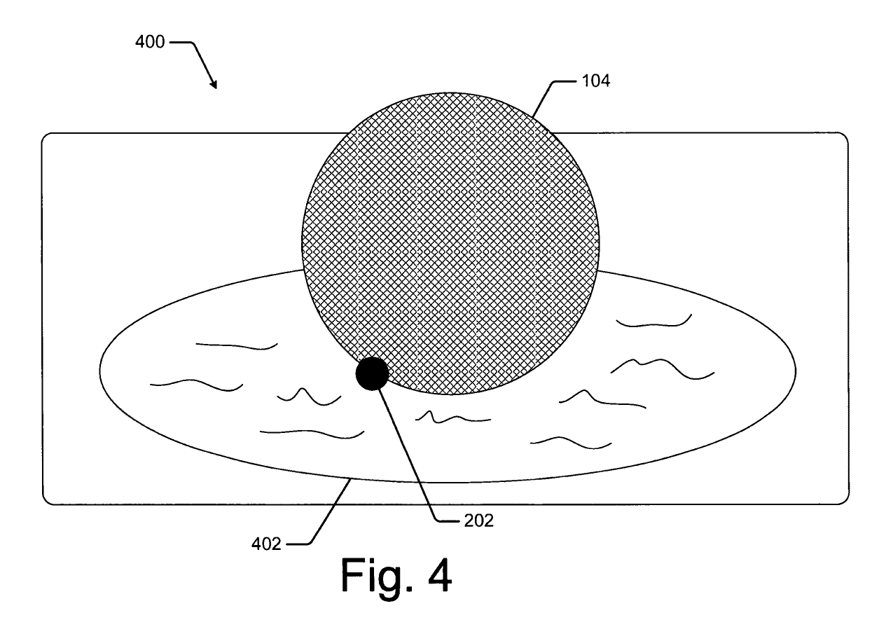

FIG. 4 is an illustration 400 of the temperature responsive polymer 108 draining from the lesion 104 once the lesion 104, and the included polymer 108, cools below the transition temperature. The tissue of interest may be breast tissue in one implementation, and the lesion 104 may be a cancerous tumor; however, the tissue 102 of interest and the lesion type could be any conceivable tissue or lesion type.

As the lesion 104 cools below the transition temperature the temperature responsive polymer 108 undergoes another phase transition from solid back to liquid. Liquid polymer 402 may drain from the tissue 102. Once in a liquid state the liquid polymer 402 can be easily separated from the excised lesion 104. After the liquid polymer 402 has completely drained, the lesion 104 retains all of its original properties.

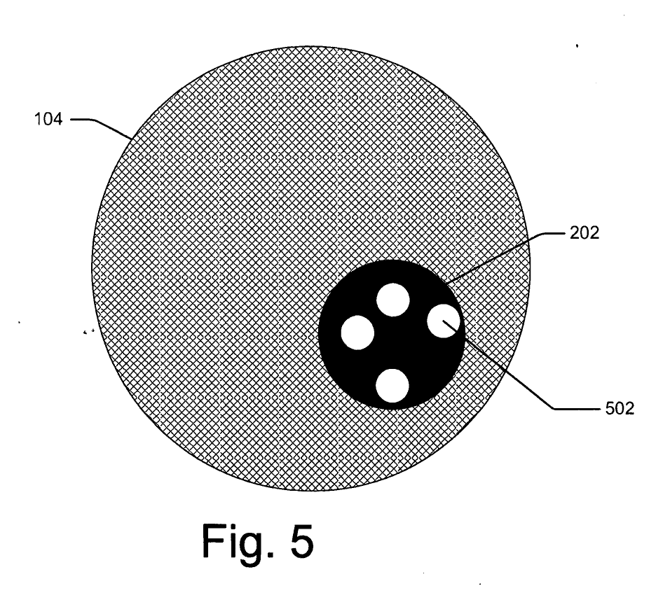

FIG. 5 shows an illustrative example 500 of an implementation of the technique illustrated in FIG. 1 in which the temperature responsive polymer 108 contains beads 502. The addition of beads 502 may also be used with the technique illustrated in FIG. 2. In the implementation shown in FIG. 1 the beads 502 in the polymer 108 can be colored to make the lesion 104 visibly distinct and in the implementation shown in FIG. 2 a boundary of the lesion 104 can be visibly distinguishable from the surrounding tissue by the presence of colored polymer 108 around the lesion 104. Additionally or alternatively, the polymer itself may be colored with a dye such as methylene blue to increase intraoperative visibility. The addition of the beads 502 may also increase the palpability of the solidified polymer 108.

In another implementation the beads 502 are made of radio opaque material so that they are visible using a technique such as radiography. In another implementation the beads 502 may improve the palpability of the polymer 108 by making the polymer 108 more rigid when the polymer 108 has reached the transition temperature and has undergone the phase change from liquid to solid.

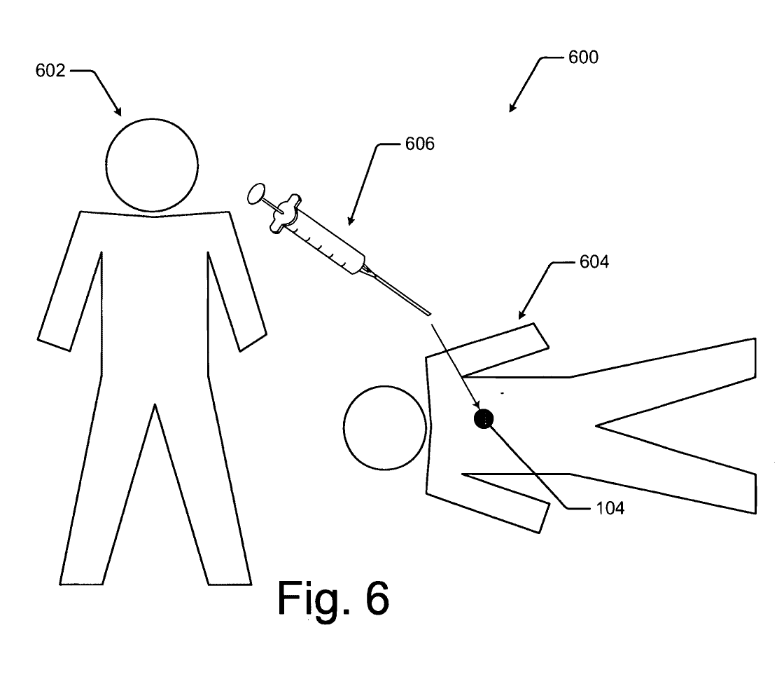

FIG. 6 is an illustrative example 600 of the techniques from FIG. 1 or FIG. 2 wherein a medical professional 602 injects a temperature responsive polymer into a patient 604. The medical professional 602 takes a syringe containing polymer 606 and injects the polymer 606 into or around a lesion 104 found in the breast (or other) tissue of the patient 604 to mark a location of the lesion 104 for excision.

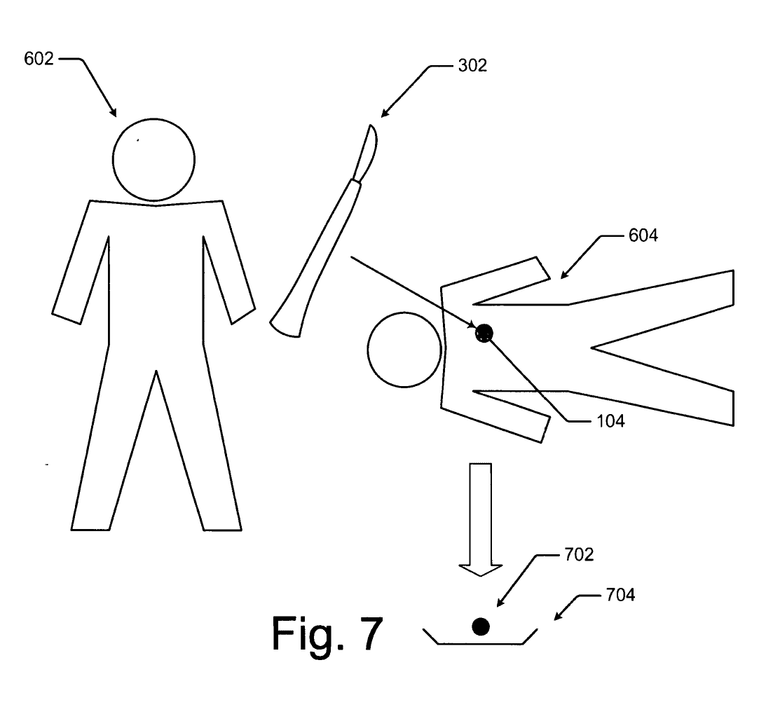

FIG. 7 is an illustrative example 700 of the techniques shown in FIG. 1 or FIG. 2 wherein a medical professional 602 removes a lesion 104 marked with a temperature responsive polymer 108 from the patient 604. The lesion 104 may be removed with a sharp or cautery dissection (e.g., the scalpel 302 as shown above in FIG. 3). The medical professional 602 uses the polymer 108 as a way to increase the palpability of the lesion 104 so to increase the chance of removing the entire lesion 104 including the margin and by so doing decreasing the likelihood that re-excision will be needed. Once removed, the excised lesion 702 can be placed in tray 704 or other container and allowed to cool to room temperature or refrigerated. The polymer 108 can be removed or drained from the excised lesion 702 after the polymer 108 returns to a liquid state. After the polymer 108 drains away from the excised lesion 702 the tissue of the lesion is unchanged from its state in situ so the excised tissue is suitable for examination because the polymer 108 does not leave artifacts.

### EXAMPLES

### Testing Polymer Reaction to Addition of Titanium Oxide Beads

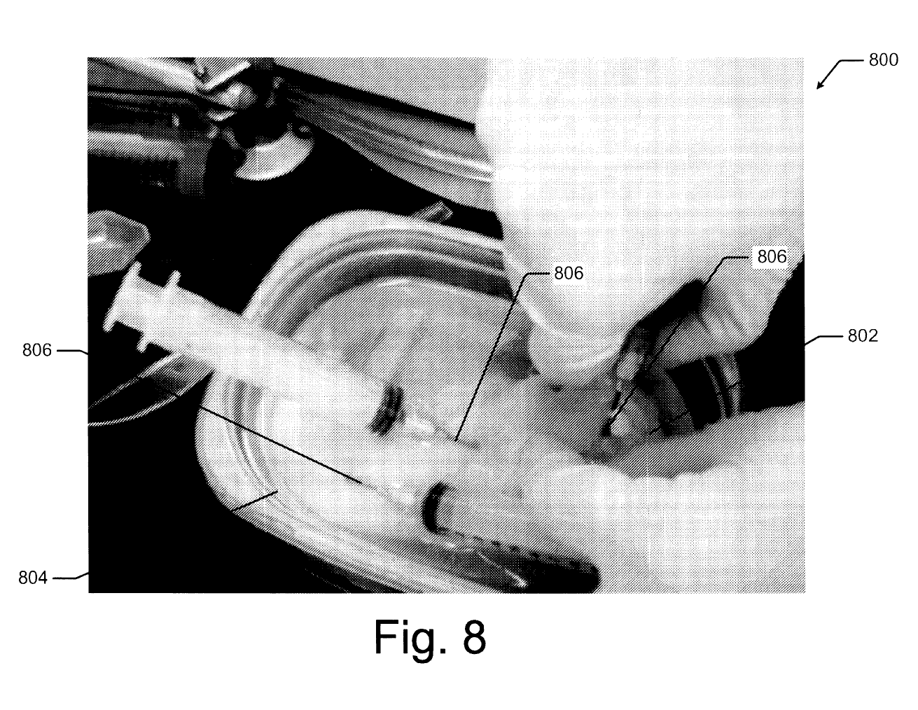

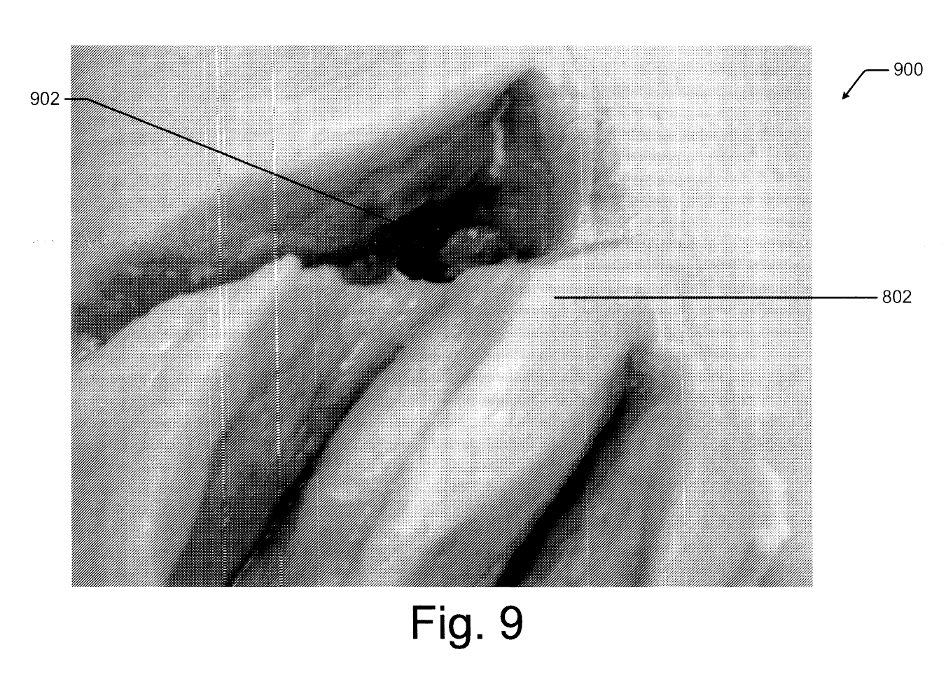

The purpose of the experiment was to determine how the addition of a radiopaque material to the hydrogel would affect the rate at which the hydrogel polymer 108 hardens in vivo. The hydrogel polymer 108 was colored with methylene blue to enhance in vivo visualization In FIGS. 8 and 9 a radiopaque material was used to examine if a polymer 108 including radiopaque beads exhibits increased palpability upon set-up. A radiopaque material in bead form may also act as a visible dye which would additionally aid in making the polymer easily detectable during surgery.

Uncooked chicken breasts 802 were used and heated to approximately 37 degrees Celsius, in a water bath 804 to simulate human breast tissue. A 16 gauge needle 806 was inserted in the breasts to inject the solutions comprising distilled water, 28% poly(N-isopropylacrylamide) and titanium oxide beads. Three different weight percentages to titanium oxide beads were tested: the first was no titanium oxide; the second was 10% titanium oxide; and, the third was 25% titanium oxide by weight. Following injection the solutions were allowed to sit for 30 minutes at 37 degrees Celsius before dissection to evaluate the effectiveness of the titanium oxide bead addition.

100411 FIG. 9 illustrates the solution, comprising the distilled water, colored polymer, and radiopaque beads, following injection of the solution, a 30 minute wait to allow the solution to harden, and incision into the chicken breast tissue 802. Incision into the chicken breast tissue 802 exposes the hardened polymer 902 for inspection of the injected solution. The methylene blue coloration of the hardened polymer 902 provided good contrast relative to the chicken breast tissue 802.

None of the injected solutions were palpable from the outside of the chicken breast tissue 802, but owning to the thickness and density of chicken breast tissue 802 this was not entirely unexpected. Human breast tissue is less dense than chicken breast so it is possible that the hardened polymer 902 may be externally palpable in a human breast. After the 30 minute wait the chicken breast tissue 802 was cut open to examine the injected polymer 902. The first two solutions (i.e., 0% titanium oxide and 10% titanium oxide) polymerized 902 and were palpable, but the third solution (i.e., 25% titanium oxide) failed to completely solidify and ultimately returned to its liquid state before it could be examined.

The hardened polymer 902 resulting from the first solution permeated the tissue within the breast to the size of a 1×0.5-0.25 inch (2.54×1.25-0.64 centimeter) rectangle. It is also possible to leave a trail of polymer leading to the injection site by continuing to inject the polymer as the needle is withdrawn. The second and third solutions returned to their liquid state before it was possible to take any size or permeability measurements.

This experiment shows that addition of titanium oxide beads at higher concentrations may have a negative effect on polymerization at body temperature. It follows then that if titanium oxide beads are to be used with poly(N-isopropylacrylamide) in distilled water, a weight percent of 10% or less should be used.

### CONCLUSION

Although the subject matter has been described in language specific to structural features and/or methodological acts, it is to be understood that the subject matter defined in the appended claims is not necessarily limited to the specific features or acts described. It will be appreciated that, based on the teachings of the present disclosure, a variety of alternate implementations may be conceived, and that the present disclosure is not limited to the particular implementations described herein and shown in the accompanying figures. Rather, the specific features and the acts are disclosed as exemplary forms of implementing the claims.

What is claimed is:

1. A method of marking a cancerous lesion in a breast to provide a medical professional visual and tactile guidance to the cancerous lesion, the method comprising:
imaging the breast with mammography to identify a location of the cancerous lesion;
inserting a needle into the breast, a tip of the needle at the location of the cancerous lesion;
injecting liquid poly(N-isopropylacrylamide) cooled below a temperature of the breast through the needle into the breast;
removing the needle;
ceasing mammography; and
surgically removing the cancerous lesion from the breast by using the hardened poly(N-isopropylacrylamide) as a reference point.

2. A method comprising;
identification of a lesion in a patient through an imaging technique;
marking a location of the lesion during the imaging technique using a temperature responsive polymer injected through an epidermis directly into a tissue of the patient.

3. The method of claim 2, wherein the lesion comprises a cancerous tumor, breast tumor, ductal carcinoma in situ, invasive/infiltrating ductal carcinoma, invasive/infiltrating lobular carcinoma, lobular carcinoma in situ, or a recurrent breast cancer.

4. The method of claim 2, wherein the imaging technique comprises mammography, sonography, magnetic resonance imaging, breast specific gamma imaging, or thermography.

5. The method of claim 2, wherein the tissue comprises tissue surrounding the lesion.

6. The method of claim 2, wherein the tissue comprises tissue of the lesion.

7. The method of claim 2, wherein the temperature responsive polymer comprises a hydrogel.

8. The method of claim 7, wherein the hydrogel comprises poly(N-isopropylacrylamide) or a poloxamer.

9. The method of claim 2, wherein the polymer undergoes a phase change from a liquid phase to a solid phase when heated above a transition temperature.

10. The method of claim 9, wherein the transition temperature is about 37 degrees Celsius.

11. The method of claim 9, wherein the phase change comprises a reversible phase change.

12. The method of claim 9, wherein the polymer in the solid phase is palpable when within the tissue.

13. The method of claim 2, wherein a tissue property of the lesion remains the same following injection of the polymer.

14. The method of claim 2, wherein the method further comprises excision of the lesion.

15. The method of claim 14, wherein the method further comprises removing the polymer from the excised lesion by allowing the excised lesion to cool below a transition temperature so that the polymer liquefies and drains from the excised lesion.

16. A kit comprising:
a temperature responsive hydrogel;
a syringe; and
a needle for delivery of the hydrogel into a tissue of a patient.

17. The kit of claim 16, wherein the temperature responsive hydrogel comprises a poloxamer.

18. The kit of claim 16, wherein the syringe is at least partially insulated.

19. The kit of claim 16, wherein the needle is insulated.

20. The kit of claim 16, further comprising a temperature controlling device.
